(19) United States
(12) Patent Application Publication
(10) Pub. No.: US 2008/0033286 A1
(43) Pub. Date: Feb. 7, 2008

(54) FIDUCIAL MARKER FOR IMAGING LOCALIZATION AND METHOD OF USING THE SAME

(75) Inventors: Willet F. Whitmore, Longboat Key, FL (US); Andrew Douglas Jones, Lake Steven, WA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: CIVCO MEDICAL INSTRUMENTS CO., INC., Kalona, IA (US)

(21) Appl. No.: 11/461,813

(22) Filed: Aug. 2, 2006

(51) Int. Cl. A61B 5/05

(52) U.S. Cl. 600/426

(57) ABSTRACT

A fiducial implant for use in the body of a living being to enable the localization, e.g., radiographic, of targeted internal tissue. The implant is in the form of an elongated, e.g., rod-like, central section and a pair of end sections, each of said end sections is arranged so that it can be readily imaged by a first type of imaging, e.g, X-ray imaging, ultrasonic imaging and magnetic resonance imaging. The implant can be inserted via a needle or cannula into targeted tissue so that when exposed to the first type of imaging the bulbous ends of the implant can be readily discerned. Once the bulbous ends are localized a therapy beam of radiation can be precisely directed to the targeted tissue

### FIELD OF THE INVENTION

This invention relates generally to fiducial markers for implantation into the body of a living being and more particularly to fiducial markers which facilitate radiographic, ultrasound or magnetic resonance imaging of such markers and methods of using the same.

### BACKGROUND OF THE INVENTION

The clinical success of focused, dose-delivery procedures, such as intensity modulated radiation therapy (IMRT) and conformal radiation therapy (CRT), is based on the accuracy of target identification and precise patient positioning. Image-guided localization is best achieved by utilizing implanted fiducial markers. In particular, such markers allow for accurate tumor localization using a variety of visualization techniques, and help these focal radiotherapy procedures by enabling real-time targeting of tumors. In addition higher doses of radiation can be targeted to the tumor and its periphery. As the trend to higher doses, smaller number of fractions, and tighter margins becomes an important part of radiotherapy, better verifiable localization is a necessity. Image-guided localization provides this type of real-time, high-precision localization. Thus, use of such markers has been deemed imperative in situations where the targeted tissue moves with respect to external marks (e.g., tattoos). In particular, prostate, liver and other such internal organs can be much more accurately targeted using implanted markers. In cases where the targeted tissue moves in an identical fashion with respect to the adjacent bony anatomy it is possible to utilize the bony anatomy as the registration points.

The Assignee of the subject invention, CIVCO Medical Solutions, of Kalona, Iowa, offers image-guided patient localization systems for localizing targeted tissue, e.g., soft tissue, such as prostate tumors, etc., under the trademark ACCULOC®. The ACCULOC® system makes use of fiducial markers in conjunction with ISOLOC™ software and electronic portal imaging (EPID), computed radiography, or standard port films to accurately locate the tissue in which the fiducial markers are implanted and thereby provide precise patient (e.g., couch) movement to achieve desired target alignment. In particular, the ISOLOC™ software algorithms provide for high-precision localization based on unique anatomic points. One of the algorithms allows the user to click on two unique points from an anterior view and a completely separate set of two points from a lateral projection. These four points are back-projected into the original 3D data-set used for planning. The program resolves the location of these points at the time of treatment and provides the couch shifts to precisely align the target. Other algorithms include single projection localization, image matching, and automated marker detection. Thus, using the ACCULOC® system markers in conjunction with the ISOLOC™ software one can easily register the target location with the treatment beam for precision dose delivery.

The markers presently sold as part of the ACCULOC® system are made of gold and are cylindrical in shape and 3 mm in length, but are available in three different diameters: 0.9 mm, 1.2 mm and 1.6 mm. The shape and size of these markers enables them to be easily inserted using a needle under ultrasound or CT guidance. The markers may be pre-loaded in needles as single markers, or as marker pairs spaced at 10 or 15 mm apart. In order to prevent migration the surface of each of the ACCULOC® soft tissue markers is specially treated, e.g., knurled.

The ISOLOC™ software algorithms provide for high-precision localization based on unique anatomic points. One of the algorithms allows the user to click on two unique points from an anterior view and a completely separate set of two points from a lateral projection. These four points are back-projected into the original 3D data-set used for planning. The program resolves the location of these points at the time of treatment and provides the couch shifts to precisely align the target. Other algorithms include single projection localization, image matching, and automated marker detection.

In U.S. patent application Ser. No. 11/422,872, filed on Jun. 7, 2006, entitled Integrated Real-Time Localization Platform Using Kilovoltage X-Rays, which is assigned to the same assignee as the subject invention and whose disclosure is incorporated by reference herein, there is disclosed and claimed a system and method for patient organ localization using fiducial markers. That system includes a kilovoltage source, a real-time marker detector and a robotic table top. The application also discloses apparatus for organ motion simulation including a motion platform controller, motion control actuators and graphical user interface for controlling the motion platform controller.

Other fiducial markers are currently available from the following companies. Best Industries, W. E. Mowrey Company, Alpha-Omega Services, Inc., and RadioMed Corporation. For example, Best Industries offers a similar marker to the markers of the ACCULOC® system, except that the Best Industries marker is not knurled. W. E. Mowrey Company, has provided markers in the form of cut-up gold wire sections of approximately 1.2×3.0 mm. Alpha-Omega Services, Inc. sells gold markers. RadioMed Corporation sells linear fiducial soft tissue markers used to localize organs, tumors and tumor beds for image-guided radiation therapy under the trade designation VISICOIL. The VISICOIL markers are in the form of an elongated helical gold coils.

The prior art also includes patents disclosing fiducial markers and method of using the same, such as United States Letters Patent Nos.: 5,397,329 (Allen) and 6,694,168 (Traxel et al.).

While the aforementioned prior art fiducial markers may be generally suitable for their intended purposes, they nevertheless leave something to be desired from one or more standpoints, e.g., requiring plural needle “sticks” to insert plural markers so that they can be imaged to provide location information with respect to all three dimensional directions, resistance to migration, and simplicity of construction.

### SUMMARY OF THE INVENTION

In accordance with one aspect of this invention there is provided a fiducial marker for use in the body of a living being. The marker comprises an elongated, e.g., rod-like, central section and a pair of end sections. Each of the end sections is of a bulbous, e.g., sphere, shape that is readily imaged by a first type of imaging, e.g., X-ray imaging, ultrasonic imaging or magnetic resonance imaging. The fiducial implant is arranged to be inserted via a needle or cannula into targeted tissue in the body of the living being, whereupon when implanted in the targeted tissue and exposed to the first type of imaging, the bulbous end sections of the implant can be readily discerned in the targeted tissue.

In accordance with one preferred aspect of this invention the elongated central section is not readily imaged by the first type of imaging, whereupon when the implant is implanted in the targeted tissue and exposed to the first type of imaging the bulbous ends of the implant can be readily discerned in the targeted tissue, with the central section being less discernable in the targeted tissue.

In accordance a method aspect of this invention, the fiducial marker is used to localize the targeted tissue. To that end, the method entails providing a fiducial implant comprising an elongated central section and a pair of end sections. Each of the end sections is of a bulbous shape that can be readily imaged by a first type of imaging, e.g., X-ray imaging, ultrasonic imaging or magnetic resonance imaging. The fiducial implant is inserted into the targeted tissue by use of a cannula or needle. Once inserted, the targeted tissue with fiducial implant therein is exposed to the first type of imaging, whereupon the bulbous end sections of the implant can be readily discerned in the targeted tissue.

In accordance with a preferred aspect of the method of this invention the location of the bulbous end sections of the implant are used to direct a therapy beam of radiation to the targeted tissue.

### DESCRIPTION OF THE DRAWING

FIG. 1 is an greatly enlarged plan view of one exemplary embodiment of a fiducial marker constructed in accordance with this invention;

FIG. 2 is an enlarged longitudinal sectional view of the distal end of a typical needle for inserting fiducial markers and shown with the fiducial marker of the subject invention loaded therein and ready for insertion into tissue to be targeted;

FIG. 3 is a greatly enlarged isometric view of one portion of an alternative fiducial marker of the subject invention.

### DETAILED DESCRIPTION OF THE PREFERRED EMBODIMENT

As will be appreciated by those skilled in the art, there are several key considerations with respect to implanted fiducial markers. In particular, the markers must be clearly visible in the radiotherapy planning image study, typically accomplished by computer tomography (CT). The markers must also be clearly visible with the treatment machine imager, whether that be electronic portal imager, x-ray film or computerized radiograph. In addition the markers must not disrupt or distort any of the aforementioned imaging modalities. Since the markers are implanted in tissue or bone they must be formed biocompatible materials. Lastly, and perhaps most importantly, the markers must remain (relatively) stable with respect to the target location and to each other from the time of treatment planning imaging study and there after until treatment is completed in order to ensure that the target tissue can be precisely located with respect to all three dimensional directions.

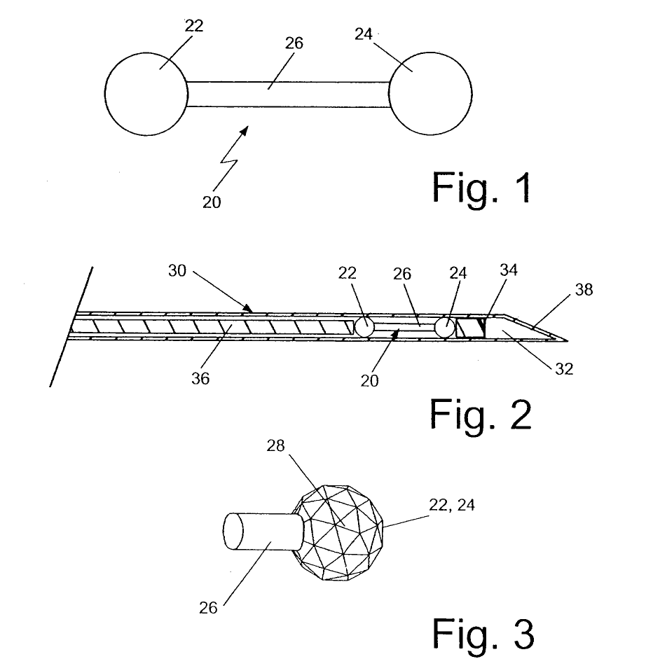

Referring now to the various figures of the drawing wherein like reference characters refer to like parts, there is shown at 20 in FIG. 1 a fiducial marker that meets the aforementioned design criteria for such devices. Thus, as will be described in detail hereinafter, the subject fiducial markers of this invention achieve all of the above design criteria, by being made up of two spaced apart portions which can be imaged by a first type of energy, e.g., radiographically imaged by X-rays, whereupon that shape not only inhibits migration of the marker itself but maintains a fixed, known distance between the two spaced apart portions. The subject fiducial markers can be used in any procedure and with any systems heretofore making use of fiducial markers. Thus, the fiducial markers of this invention can be used in systems and with methods like those disclosed in copending application Ser. No. 11/422,872.

The marker 20 is of an overall “dumbbell” like shape and basically comprises a pair of end sections 22 and 24 which are each of a bulbous shape and which are interconnected by an elongated central section 26. In the preferred embodiment the bulbous shape of each end section is a sphere. However, it is contemplated that other shapes can be used as well, such as ovoids, cylinders, etc. Each end section is formed of a biocompatible material that it can be readily imaged and discerned by some type of imaging modality, e.g., X-ray imaging, ultrasound or magnetic resonance imaging. For example, as is known to those skilled in the art, for X-ray imaging it is the atomic weight/density/volume of the marker to be imaged that makes it visible and thus the material/volume of the bulbous end sections 26 of this invention are essential for visibility. In the exemplary preferred embodiment, where the imaging technology is X-ray, the spheres are formed of gold. However, other suitable materials can be used to form the spheres. Moreover, the spheres may be formed of one biocompatible material and coated with another biocompatible material providing that the resultant combination provides the desired atomic weight/density/volume for visibility by X-ray imaging. For ultrasonic imaging, the spheres may be formed of an echogenic material or coated with an echogenic material. Angiotech Pharmaceuticals, Inc. offers a suitable echogenic coating under the trademark ECHO-COAT®. For magnetic resonance imaging it is the electrical polarity/conductivity of the material and it's disturbance by RF signals that affects visibility, and volume rather than surface is essential here as well. Thus, in accordance with one preferred embodiment of this invention for magnetic resonance imaging, the spheres are formed of a non-conductive metal, e.g., titanium, or formed of any other suitable biocompatible material and coated with a non-conductive metal providing that the resultant combination provides the desire electrical polarity/conductivity and volume for visibility under magnetic resonance imaging.

As mentioned above, the spheres are connected together by an elongated central bridging section 26. In the preferred exemplary embodiment shown, the elongated bridging section is a rod-like member which is formed of a biocompatible material that is not readily imaged by the imaging modality used, so that the bridging section is considerably less discernable than the bulbous ends when the fiducial marker is imaged. For X-ray imaging applications the central bridging section 26 is preferably formed of a titanium or other biocomptible materials having a significantly lower density than gold so that it is is considerably less discernable, e.g., almost invisible, when imaged. For magnetic resonance imaging applications the elongated central section 26 may be formed of a biocompatible plastic. Accordingly, when the fiducial implant 20 is in position in the tissue to be localized, only the spheres 22 and 24 will be visible. This action effectively create a pair of points that can be used with the associated software, like the ISOLOC™ software, to help localize the marker. For most applications two fiducial markers will be implanted, thereby creating four discrete localizable points. The software may only need to utilize three of those points to localize the targeted tissue. In any case, in order to ensure precise localization of the targeted tissue it is of considerable importance that the distance between the two bulbous ends 22 and 24 of the fiducial marker 20 be fixed and maintained at that fixed, consistent distance from each other when the marker is implanted. Accordingly, the central section 26 of the fiducial marker is substantially rigid so that it doesn't flex or otherwise deform, which action could change the spacing between the bulbous ends of the marker. Moreover, the fixed distance between the spheres can be stored in the software for verification purposes.

In accordance with a preferred embodiment of this invention for use by X-ray imaging the ends 22 and 24 are formed of gold and each has a diameter in the range of approximately 0.5 mm to 3 mm, with the most preferred diameter being approximately 1.2 mm. The elongated rod-like central section 26 is formed of titanium and is of a diameter of approximately 0.2 mm to 2 mm, with the most preferred diameter being approximately 0.2 mm. The length of the rod-like member 26 is in the range of approximately 5 mm to 30 mm, with the most preferred length being approximately 10 mm.

As should be appreciated by those skilled in the art, the general overall “dumbbell” shape of the fiducial marker 20 tends to makes it quite resistant to migration. If further resistance to migration is desired, the surface of the marker may be textured. In particular, as shown in FIG. 3 the outer surface of each of the bulbous ends 22 and 24 may be knurled 28 or otherwise roughened.

The markers of this invention are arranged to be loaded into a needle or cannula 30, like shown schematically in FIG. 3, for delivery into the tissue to be targeted, e.g., the prostate. To that end one or more markers 20 constructed in accordance with this invention is disposed within the hollow central lumen 32 in the needle just proximally of a removable plug 34. The plug is formed of a biologically inert sterile wax and serves to hold the marker(s) in place in the lumen 32 between it and a pusher rod 36. The sharpened distal end 38 of the needle is directed into the targeted tissue and the pusher rod actuated to push the plug 34 and the marker 20 out of the lumen and thereby deposit the plug and marker in the targeted tissue. If the needle contains a second marker the needle can be retracted and then introduced into a second site in the targeted tissue to deposit the second marker thereat. If the needle only contains one marker 20 another needle will be required to deposit the second marker 20 in the targeted tissue. Thus, using the subject invention only two “needle sticks” will be necessary to produce four discernable points for localizing the targeted tissue (although only three of such points may be necessary with the localization software), whereas prior art systems for localizing the targeted tissue would require at a minimum three needle sticks. This feature of the subject invention offers a considerable advantage over the prior art insofar as patient comfort is concerned. Moreover, as noted above the shape of the markers of this invention provide them with excellent resistance to migration and provide plural discernable imaging points which remain at a consistent fixed distance from each other.

Without further elaboration the foregoing will so fully illustrate our invention that others may, by applying current or future knowledge, adopt the same for use under various conditions of service.

We claim:

1. A fiducial implant for use in the body of a living being comprising an elongated central section and a pair of end sections, each of said end sections being of a bulbous shape that can be readily imaged by a first type of imaging, said fiducial implant being arranged to be inserted via a needle or cannula into targeted tissue in the body of the living being, whereupon when implanted in the targeted tissue and exposed to the first type of imaging the bulbous ends of the implant can be readily discerned in the targeted tissue.

2. The fiducial implant of claim 1 wherein said elongated central section is not readily imaged by said first type of imaging, whereupon when said implant is implanted in the targeted tissue and exposed to said first type of imaging said bulbous ends of the implant can be readily discerned in the targeted tissue, with the central section being less discernable in the targeted tissue.

3. The fiducial implant of claim 1 wherein said first type of imaging is X-ray imaging and wherein the atomic weight/density/volume each of said bulbous end sections renders them visible under X-ray imaging, whereupon said each of said bulbous end sections can be readily discerned by X-ray imaging.

4. The fiducial implant of claim 2 wherein said first type of imaging is X-ray imaging and wherein the atomic weight/density/volume of each of said bulbous end sections renders them visible under X-ray imaging, whereupon said each of said bulbous end sections can be readily discerned by X-ray imaging.

5. The fiducial implant of claim 1 wherein said first type of imaging is ultrasonic imaging and wherein each of said bulbous end sections has a surface which renders it visible under ultrasonic imaging, whereupon each of said bulbous end sections can be readily discerned by ultrasonic imaging.

6. The fiducial implant of claim 2 wherein said first type of imaging is ultrasonic imaging and wherein each of said bulbous end sections has a surface which renders it visible under ultrasonic imaging, whereupon each of said bulbous end sections can be readily discerned by ultrasonic imaging.

7. The fiducial implant of claim 1 wherein said first type of imaging is magnetic resonance imaging and wherein the electrical polarity/conductivity and volume of each of said bulbous end sections renders it visible under magnetic resonance imaging, whereupon said each of said bulbous end sections can be readily discerned by magnetic resonance imaging.

8. The fiducial implant of claim 2 wherein said first type of imaging is magnetic resonance imaging and wherein the electrical polarity/conductivity and volume of each of said bulbous end sections renders it visible under magnetic resonance imaging, whereupon said each of said bulbous end sections can be readily discerned by magnetic resonance imaging.

9. The fiducial implant of claim 3 wherein each of said bulbous end sections comprises gold.

10. The fiducial implant of claim 9 wherein said elongated central section comprises titanium.

11. The fiducial implant of claim 5 wherein said surface of each of said bulbous end sections comprises an echogenic coating.

12. The fiducial implant of claim 7 wherein each of said bulbous end sections comprises a non-conductive metal.

13. The fiducial implant of claim 12 wherein said metal is titanium.

14. The fiducial implant of claim 13 wherein said elongated central section comprises a plastic.

15. The fiducial implant of claim 1 wherein each of said bulbous end sections has an outer surface that is textured to provide the implant with resistance to migration in the targeted tissue.

16. The fiducial implant of claim 15 wherein said textured outer surface is knurled.

17. The fiducial implant of claim 1 wherein said elongated central section is substantially rigid whereupon the spacing between said bulbous end sections of said implant is maintained at a fixed distance.

18. The fiducial implant of claim 1 wherein each of said bulbous end sections is a sphere.

19. The fiducial implant of claim 18 wherein each of said spheres has a diameter is in the range of approximately 0.5 mm to 3 mm.

20. The fiducial implant of claim 19 wherein said diameter is approximately 1.2 mm.

21. The fiducial implant of claim 1 wherein said elongated central section is a rod.

22. The fiducial implant of claim 21 wherein said rod has a diameter in the range of approximately 0.2 mm to 2 mm.

23. The fiducial implant of claim 22 wherein said diameter is approximately 0.2 mm.

24. The fiducial implant of claim 22 wherein said rod is in the range of approximately 5 mm to 30 mm long.

25. The fiducial implant of claim 20 wherein said rod is approximately 10 mm long.

26. A method of localizing targeted tissue within the body of a living being, said method comprising:
providing a fiducial implant comprising an elongated central section and a pair of end sections, each of said end sections being of a bulbous shape that can be readily imaged by a first type of imaging;
inserting said fiducial implant into the targeted tissue by use of a cannula or needle; and
exposing said targeted tissue with said fiducial implant inserted therein to said first type of imaging, whereupon the bulbous end sections of said implant can be readily discerned in the targeted tissue.

27. The method of claim 26 wherein said first type of imaging is X-ray imaging.

28. The method of claim 26 wherein said first type of imaging is ultrasonic imaging.

29. The method of claim 26 wherein said first type of imaging is magnetic resonance imaging.

30. The method of claim 27 wherein said elongated central section of said implant is not readily imaged by said first type of imaging.

31. The method of claim 28 wherein said elongated central section of said implant is not readily imaged by ultrasonic imaging.

32. The method of claim 29 wherein said elongated central section of said implant is not readily imaged by magnetic resonance imaging.

33. The method of claim 27 additionally comprising utilizing the location of said bulbous end sections of the implant to direct a therapy beam of radiation to said targeted tissue.
